(19) United States
(12) Patent Application Publication
(10) Pub. No.: US 2025/0090190 A1
(43) Pub. Date: Mar. 20, 2025

(54) ULTRASONIC TRANSDUCER, ULTRASONIC TREATMENT TOOL AND METHOD OF PRODUCING ULTRASONIC TRANSDUCER

(71) Applicant: Olympus Medical Systems Corp., Tokyo (JP)

(72) Inventors: Tsubasa NIIYAMA, Tokyo (JP); Marina Asahina, Sagamihara-shi (JP)

(21) Appl. No.: 18/828,650

(22) Filed: Sep. 9, 2024

Related U.S. Application Data
(60) Provisional application No. 63/583,014, filed on Sep. 15, 2023.

(51) Int. Cl. A61B 17/32

(52) U.S. Cl. CPC A61B 17/320068; A61B 17/320092; A61B 2017/320082; A61B 2017/320094

(57) ABSTRACT

An ultrasonic transducer can include a plurality of piezoelectric elements arrayed along a longitudinal direction. The ultrasonic transducer can further include housing configured to store the plurality of piezoelectric elements and a contact receiver that is fitted to the housing and configured to transmit an electric signal to the plurality of piezoelectric elements. The contact receiver can include a body that is electrically insulating and a wiring pattern that is electrically connected to a lead that is provided on the housing. The wiring pattern can be formed by three-dimensional plating.

### CROSS-REFERENCE TO RELATED APPLICATION

This application is based on and claims priority under 35 U.S.C. § 119 to U.S. Provisional Application No. 63/583,014, filed Sep. 15, 2023, the entire contents of which are incorporated herein by reference.

### TECHNICAL FIELD

The present disclosure relates to an ultrasonic transducer, an ultrasonic treatment tool and a method of producing an ultrasonic transducer.

### BACKGROUND

An ultrasonic treatment tool is a tool that applies energy, such as ultrasound energy, to a subject region to be treated in living tissue and thereby treats the subject region. An example of such a treatment tool is described in International Publication Pamphlet No. 2011/0121827. As for the ultrasonic treatment tool according to Patent Literature 1, an ultrasound transducer that generates ultrasonic vibrations is detachable to a hand piece.

### SUMMARY

In some embodiments, an ultrasonic transducer can include: a plurality of piezoelectric elements arrayed along a longitudinal direction. The ultrasonic transducer can further include a housing configured to store the plurality of piezoelectric elements and a contact receiver that is fitted to the housing. The contact receiver can be configured to transmit an electric signal to the plurality of piezoelectric elements. The contact receiver can include a body that is electrically insulating and a wiring pattern that is electrically connected to a lead that is provided on the housing. The wiring pattern can be formed by three-dimensional plating.

In some embodiments, an ultrasonic treatment tool can include an ultrasonic transducer and a hand piece. The ultrasonic transducer can include a plurality of piezoelectric elements arrayed along a longitudinal direction. The tool can further include a housing configured to store the plurality of piezoelectric elements and a contact receiver fitted to the housing. The contact receiver can be configured to transmit an electric signal from the hand piece to the plurality of piezoelectric elements. The contact receiver can include a body that is electrically insulating and a wiring pattern that is electrically connected to a lead provided on the housing. In an example, the wiring pattern can be formed by three-dimensional plating.

In some embodiments, provided is a method of producing an ultrasonic transducer including a plurality of piezoelectric elements arrayed along a longitudinal direction. A housing configured to store the plurality of piezoelectric elements and a contact receiver that is fitted to the housing. The contact receiver being configured to transmit an electric signal to the plurality of piezoelectric elements. The method can include molding a body that is electrically insulating, forming a wiring pattern by three-dimensional plating on the body, and electrically connecting the wiring pattern and a lead provided on the housing.

The above and other features, advantages and technical and industrial significance of this disclosure will be better understood by reading the following detailed description of embodiments of the disclosure, when considered in connection with the accompanying drawings.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a diagram schematically illustrating an entire configuration of an ultrasonic treatment system according to an embodiment of the present disclosure.

FIG. 2 illustrates and example of a cross-sectional view of an ultrasonic transducer that is used in the ultrasonic treatment system in FIG. 1.

FIG. 3 illustrates an example of a diagram illustrating connection between the ultrasonic transducer and a contact unit.

FIG. 4 illustrates an example of a perspective view illustrating a configuration of a contact receiver that the ultrasonic transducer includes.

FIG. 5 illustrates an example of a cross-sectional view illustrating a configuration of the contact receiver.

FIGS. 6A to 6C illustrate an example of diagrams of a method of forming a pattern according to an embodiment of the present disclosure.

FIG. 7 illustrates an example of a cross-sectional view illustrating a configuration of a contact receiver that an ultrasonic transducer according to an embodiment of the present disclosure.

FIG. 8 illustrates an example of a diagram of a method of producing a contact receiver according to an element of the present disclosure.

FIGS. 9A and 9B illustrate example diagrams of a method of producing a contact receiver according to an embodiment of the present disclosure.

FIG. 10 is an example of a diagram of a method of producing a contact receiver according to an embodiment of the present disclosure.

FIG. 11 illustrates an example of a partial cross-sectional view illustrating a configuration of part of a contact receiver of an ultrasonic transducer according to an embodiment of the present disclosure.

FIG. 12 illustrates an example of a partial cross-sectional view illustrating a configuration of part of a contact receiver of an ultrasonic transducer according to an embodiment of the present disclosure.

### DETAILED DESCRIPTION

In the following description, ultrasonic treatment systems including an ultrasonic transducer will be described as modes for carrying out the disclosure (referred to as “embodiments” below). The embodiments do not limit the disclosure. Furthermore, in the illustration of the drawings, the same parts are denoted with the same reference numerals. Furthermore, the drawings are schematic and it is necessary to note that the correlation among members in thickness and width, the ratio of each member, etc., may differ from actual ones. Portions whose ratios differ among mutual drawings are also contained.

### Embodiment 1

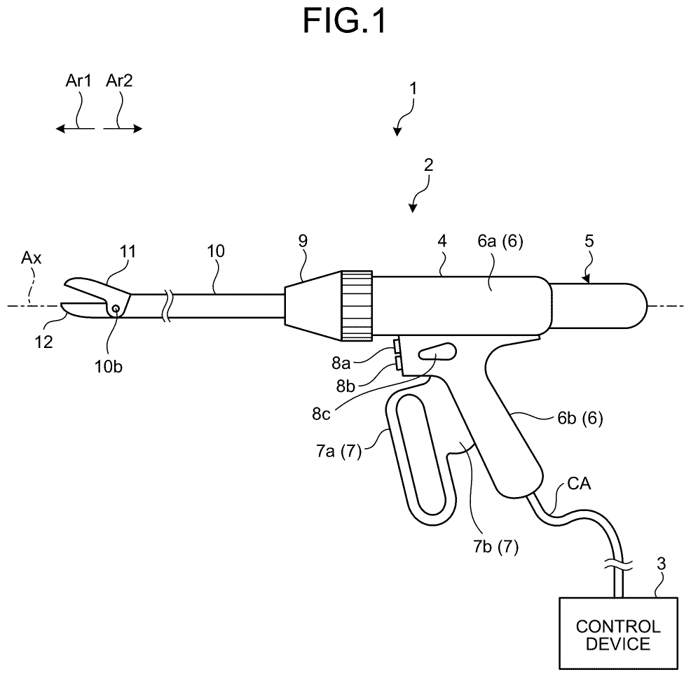

FIG. 1 is a diagram schematically illustrating an entire configuration of an ultrasonic treatment system 1 according to Embodiment 1 of the disclosure. For convenience in description, one side along a center axis Ax of a sheath 10 is referred to as a distal-end side Ar1 and the other side is referred to as a proximal-end side Ar2. The ultrasonic treatment system 1 can apply treatment energy to a region to be treated (“subject region” below) in living tissue, thereby treating the subject region. In Embodiment 1, high frequency energy can be employed as the treatment energy in addition to ultrasonic energy; however, the system may employ only ultrasonic energy. As illustrated in FIG. 1, the ultrasonic treatment system 1 can include an ultrasonic treatment tool 2 and a control device 3.

The ultrasonic treatment tool 2, can be a medical treatment tool that can treat a subject region with the tool penetrating through an abdominal wall. The ultrasonic treatment tool 2 can include a hand piece 4 and an ultrasonic transducer 5.

The hand piece 4 can include a holder case 6, a movable handle 7, a first switch 8a, a second switch 8b, third switches 8c in a pair, a rotation knob 9, a sheath 10, a jaw 11, an ultrasonic probe 12, and a cable CA.

The holder case 6 can support the entire ultrasonic treatment tool 2. The holder case 6 can include a holder case body 6a with an approximately cylindrical shape and that is coaxial with the center axis Ax. The holder case can further include a fixed handle 6b that extends from the holder case body 6a to a lower side and that is gripped by an operator, such as a practitioner.

The movable handle 7 can receive each of a close operation and an open operation performed by the operator, such as a practitioner. When the close operation is received, the movable handle 7 can move in a direction in which the movable handle 7 gets close to the fixed handle 6b. On the other hand, when the open operation on the movable handle 7 is received, the movable handle 7 can move in a direction in which the movable handle 7 separates from the fixed handle 6b.

Each of the first and second switches 8a and 8b is provided in a state of being exposed to the outside from a side surface on the distal end side Ar1 in the fixed handle 6b. The first switch 8a can receive an operation in a first energy output mode performed by the operator. The second switch 8b can receive an operation of setting a second energy output mode performed by the operator. The second energy output mode can be an energy output mode different from the first energy output mode.

An example of the first energy output mode can include an energy output mode in which coagulation and incision are performed on a subject region by applying ultrasonic energy and high frequency energy. An example of the second energy output mode can be an energy output mode in which coagulation is performed on a subject region by applying high frequency energy.

The third switches 8c in a pair are provided on a front side and a back side on the drawing (not illustrated in the drawing) in a state of being exposed to the outside of the fixed handle 6b. The third switches 8c in a pair can receive an operation of a third energy output mode performed by the operator. An example of the third energy output mode can include an energy output mode in which coagulation and incision are performed on a subject region by applying ultrasonic energy, or the like.

The rotation knob 9 can have or be formed in an approximately cylindrical shape that is coaxial with the center axis Ax and can be provided on the distal-end side Ar1 of the holder case body 6a. The rotation knob 9 can receive a rotation operation performed by the operator. Because of the rotation operation, the rotation knob 9 can rotate on the center axis Ax with respect the holder case body 6a. Because of the rotation of the rotation knob 9, the jaw 11 and the ultrasonic probe 12 can rotate on the center axis Ax.

The sheath 10 can have or be formed in an approximately or substantially cylindrical shape. An end of the sheath 10 on the proximal end side Ar2 can be attached to the holder case body 6a.

The jaw 11 can grip the subject region between the jaw 11 and the end of the ultrasonic probe 12 on the distal end side Ar1. An open-close mechanism that can cause the jaw 11 to open or close with respect to the end of the ultrasonic probe 12 on the distal-end side Ar1 according to the open-close operation performed by the practitioner on the movable handle 7 can be provided in the holder case body 6a and the sheath 10 that are described above.

The ultrasonic probe 12 can be made of an electrically conducting material and can have or be formed in an elongated shape that extends linearly along the center axis Ax. The ultrasonic probe 12 can be inserted into the sheath 10 with its end on the distal-end side Ar1 protruding to the outside. The end of the ultrasonic probe 12 on the proximal end side Ar2 can be mechanically connected to the ultrasonic transducer 5 described below. In other words, the ultrasonic transducer 5 can rotate on the center axis Ax together with the ultrasonic probe 12 according to the rotation operation performed by the operator on the rotation knob 9. The ultrasonic probe 12 can transmit ultrasonic vibrations generated by the ultrasonic transducer 5 from the end on the proximal end side Ar2 to the end on the distal end side Ar1. In the present embodiment, the ultrasonic vibrations can include longitudinal vibrations that are vibrations along the center axis Ax.

The cable CA can be detachably connected to the control device 3. Thus, the cable CA can be electrically connected to the control device 3.

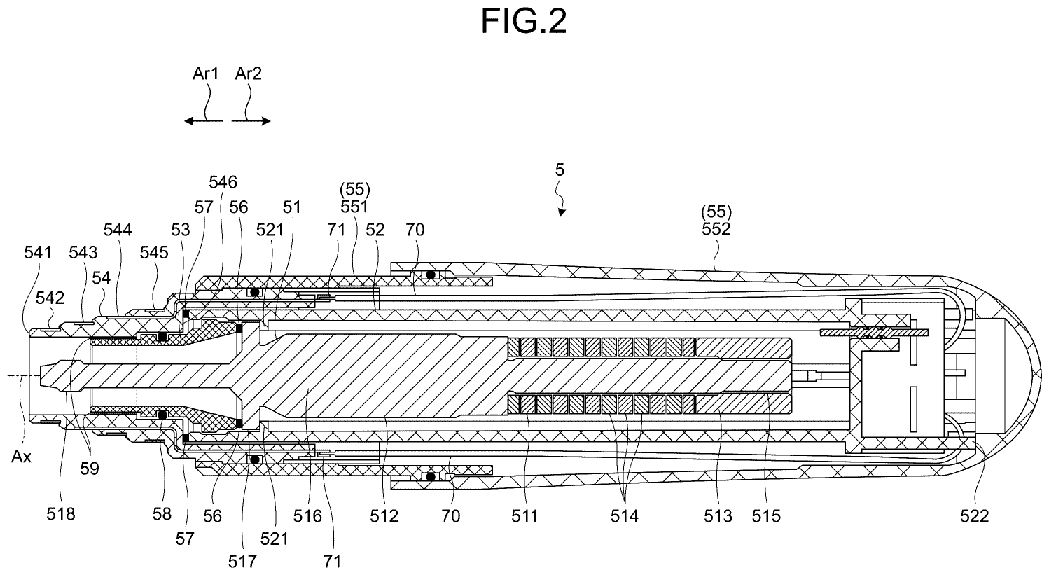

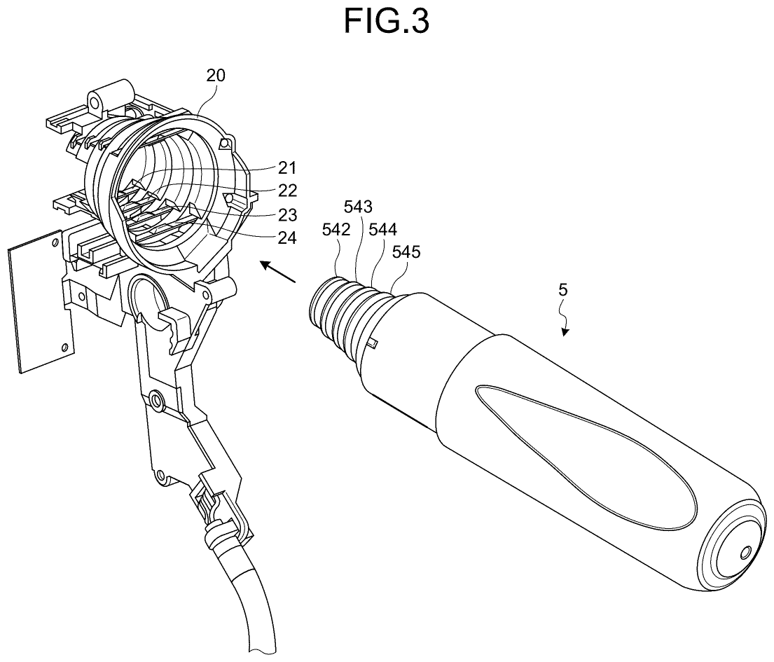

FIG. 2 is an example of a cross-sectional view of the ultrasonic transducer 5 that is used in the ultrasonic treatment system 1 in FIG. 1. FIG. 3 is an example of a diagram illustrating connection between the ultrasonic transducer 5 and a contact unit.

The ultrasonic transducer 5 includes an ultrasound transducer portion 51, a storage 52, a holder 53, a contact receiver 54, and a casing 55.

Under the control of the control device 3, the ultrasound transducer portion 51 can generate ultrasound vibrations. In Embodiment 1, the ultrasound transducer portion 51 can include a BLT (bolt-clamped Langevin type transducer). The ultrasound transducer portion 51 can include a transducer body 511, a front mass 512, and a back mass 513.

The transducer body 511 can include a plurality of piezoelectric elements 514. The piezoelectric elements 514 each have a disc-like shape and can include an opening at the center and are layered along the center axis Ax. The piezoelectric elements 514 can alternately repeat expansion and contraction along the direction of the layering because a difference in potential occurs in the direction of the layering along the center axis Ax according to a control signal supplied to an electrode plate. Accordingly, the ultrasound transducer portion 51 can generate ultrasonic vibrations that are longitudinal vibrations in which the direction of the layering is the direction of the vibrations.

The front mass 512 can be made of an electrically conducting material and can have or be formed in an elongated shape that extends linearly along the center axis Ax. The front mass 512 can include an element attachment portion 515, a cross-sectional area change portion 516, a flange portion 517, and a probe attachment portion 518.

The element attachment portion 515 can include a bolt that extends linearly along the center axis Ax and can be inserted into each of openings of a plurality of electrode plates and each of openings of the piezoelectric elements 514. The back mass 513 can include a nut made of an electrically conducting material connected to an end of the element attachment portion 515 on the proximal end side Ar2.

The cross-sectional area change portion 516 can include a portion provided at an end of the element attachment portion 515 on the distal end side Ar1 and that can amplify the amplitude of ultrasonic vibrations. In the cross-sectional area change portion 516, a diameter size of an end of on the proximal end side Ar2 can be set larger than that of the element attachment portion 515 and an end on the distal end side Ar1 can have a shape of a circular truncated cone whose cross-sectional area decreases toward the distal end side Ar1. In other words, the piezoelectric elements 514 can be interposed between the cross-sectional area change portion 516 and the back mass 513 with the element attachment portion 515 penetrating the piezoelectric elements 514 along the center axis Ax and thus can be integrally fastened in a state of having the approximately cylindrical shape.

The flange portion 517 can be provided at an end of the cross-sectional area change portion 516 on the distal-end side Ar1. A diameter size of the flange portion 517 can be set larger than that of the cross-sectional area change portion 516. The flange portion 517 can be interposed between a protrusion 521 of the storage 52 to be described below and the holder 53. A seal ring 56 can be arranged between the flange portion 517 and the holder 53. Because of fixation of the flange portion 517 by the storage 52 and the holder 53, the position of the ultrasound transducer portion 51 in the axial direction can be fixed.

The probe attachment portion 518 can be provided at an end of the flange portion 517 on the distal-end side Ar1 and can extend linearly along the center axis Ax.

The storage 52 can be made of a resin material that is an electrical insulator, can have a cylindrical shape, and can store the ultrasound transducer portion 51. The casing 55 can be a housing made of a resin material that is an electrical insulator, can have a tubular shape, and with which the outer side of the storage 52 can be covered. The casing 55 can consist of a first casing part 551 that can be tubular and that can be positioned on the distal end side Ar1 and a second casing part 552 that can be tubular, that can have a bottom, and that can be positioned on the proximal end side Ar2.

An end 522 of the storage 52 on the proximal end side Ar2 can be pushed against an inner wall of the second casing part 552 of the casing 55. The holder 53 can be made of a resin material that is an electrical insulator and can be tubular. The proximal end side Ar2 of the holder 53 can be inserted into the distal end side Ar1 of the storage 52. The distal end side Ar1 of the holder 53 can be inserted into the proximal end side Ar2 of the contact receiver 54 to be described below.

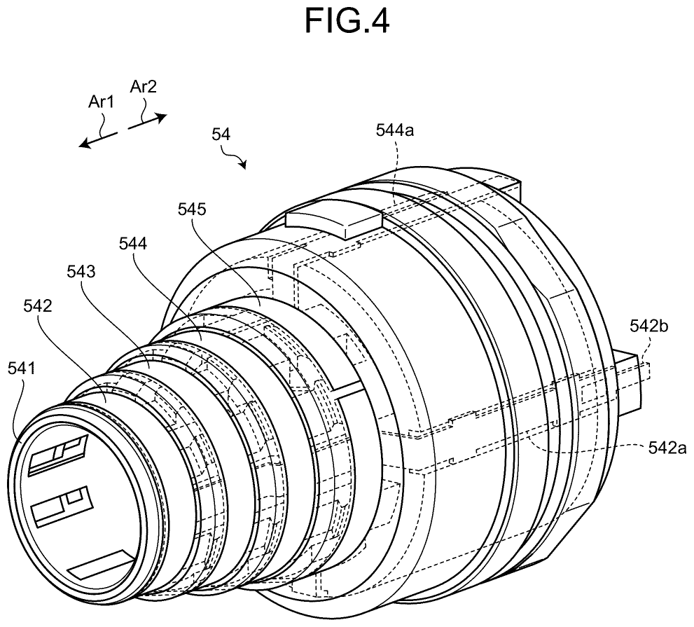

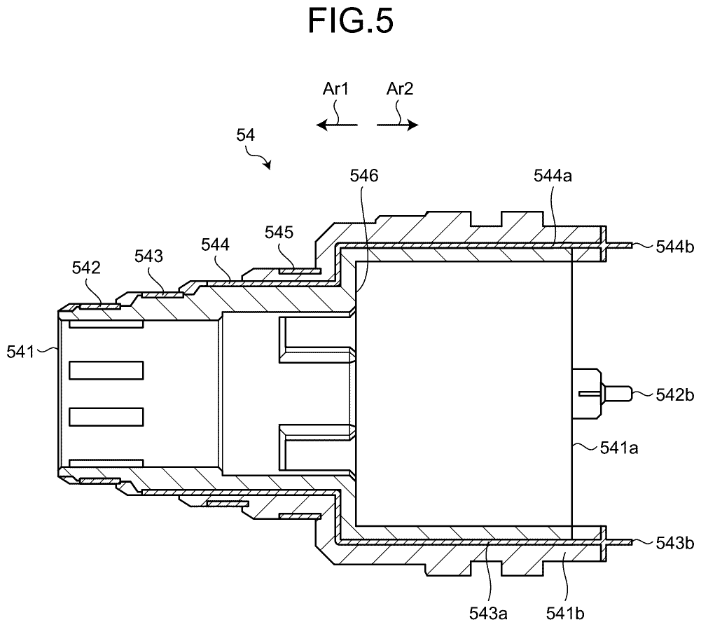

FIG. 4 illustrates an example of a perspective view illustrating a configuration of a contact receiver that the ultrasonic transducer includes. FIG. 5 illustrates an example of a partial cross-sectional view illustrating the configuration of the contact receiver that the ultrasonic transducer includes. The contact receiver 54 can be cylindrical and can include a body 541 whose outer diameter can increase in steps from the distal end side Ar1 to the proximal end side Ar2. The body 541 can be an electrical insulator and, for example, can be made of a resin material.

The contact receiver 54 can be attached to the casing 55. Specifically, the distal end side Ar2 of the contact receiver 54 can be inserted into the casing 55. The distal end side Ar1 of the storage 52 can be inserted into the proximal end side Ar2 of the contact receiver 54 and a rubber material 57 can be arranged between a step portion 546 on an inner circumference of the contact receiver 54 and the distal end side Ar1 of the storage 52 that is pushed. Furthermore, a cable 70 that can transmit power and control signals to the ultrasound transducer portion 51 can be connected to the proximal end side Ar2 of the contact receiver 54.

A first contact portion 542, a second contact portion 543, a third contact portion 544, and a fourth contact portion 545 can be formed in the respective step portions. The first contact portion 542, the second contact portion 543, the third contact portion 544, and the fourth contact portion 545 can have ring shapes. Each contact portion can be made of an electrically conducting material and can be provided entirely around the respective step portions in the circumferential direction. In each of the contact portions, a pattern extending to the proximal end side Ar2 is formed. This pattern can be formed of an electrically conducting material. Specifically, a first pattern 542a can be connected to the first contact portion 542. The first pattern 542a can be connected to a lead 71 that extends from the cable 70 at an end 542b on a side opposite to the side of the first contact portion 542 of the first pattern 542a. A second pattern 543a can be connected to the second contact portion 543 and the second pattern 543a can be connected to the cable 70 (the lead 71) at an end 543b of the second pattern 543a on a side opposite to the side of the second contact portion 543. A third pattern 544a can be connected to the third contact portion 544 and the third pattern 544a can be connected to the cable 70 (the lead 71) at an end 544b of the third pattern 544a on a side opposite to the side of the third contact portion 544. A fourth pattern 545a can be connected to the fourth contact portion 545 and the fourth pattern 545a can be connected to the cable 70 (the lead 71) at an end 545b of the fourth pattern 545a on a side opposite to the side of the fourth contact portion 545. Each of the patterns corresponds to a wiring pattern.

The body 541 can include a first portion 541a and a second portion 541b. Each of the first portion 541a and the second portion 541b can be formed using a resin material. For example, super engineering plastic, such as PEEK (Poly Ether Ether Ketone), PEKK (Poly Ether Ketone Ketone), or PPSU (Poly Phenyl Sulfone), can be used as the resin material.

In the body 541, the second portion 541b can cover an outer surface of the first portion 541a and can expose the contact portions (the first contact portion 542, the second contact portion 543, the third contact portion 544, and the fourth contact portion 545).

The contact receiver 54 can be made by molding the body 541 and forming a pattern on the body 541. Making the contact receiver 54 can include molding the body 541 and forming a pattern by three-dimensional plating on the body 541. Due to electrically connecting the pattern (for example, the end 542b) of the contact receiver 54 and the lead 71 that is provided in the casing 55, the contact receiver and the casing 55 can be electrically connected and the ultrasonic transducer 5 is made.

Specifically, the contact receiver 54 can be made by performing pattern formation on a surface of the first portion 541a after molding the first portion 541a (primary molding), then molding the second portion 541b (secondary molding and fitting the contact portions together.

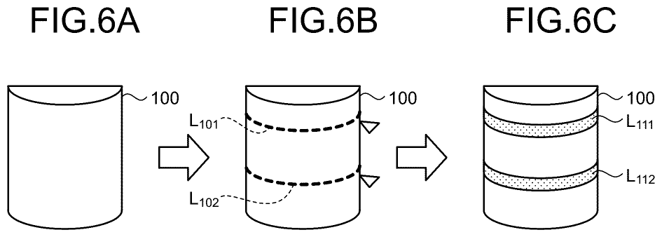

FIGS. 6A to 6C illustrate example diagrams of a method of forming a pattern in Embodiment 1 of the disclosure. In pattern formation, for example, laser can be applied to a side surface of a resin portion 100 (refer to FIG. 6A) in pattern formation positions L₁₀₁ and L₁₀₂ (refer to FIG. 6B). Thereafter, patterns L₁₁₁ and L₁₁₂ can be formed by, for example, electroless plating in the positions of the application of laser (refer to FIG. 6C). At that time, three-dimensional plating in which patterns are formed three-dimensionally can be performed on a resin compact. It is possible to form first to fourth patterns 542a to 545a as three-dimensional plating using this pattern forming method. The thickness of the pattern can be, for example, approximately few μm. The compact made by this pattern forming method is referred to as a MID (Molded Interconnect Device) as one in which an electric circuit can be formed on a surface of the resin mold having a three-dimensional shape.

After the pattern is formed, the first contact portion 542, the second contact portion 543, the third contact portion 544, and the fourth contact portion 545 can be fitted to the surface of the body 541 and accordingly the first contact portion 542, the second contact portion 543, the third contact portion 544, and the fourth contact portion 545 can be fitted to the body 541.

Back to FIG. 2 and FIG. 3, a contact unit 20 can be provided in the holder case body 6a and, when the ultrasonic transducer 5 is connected to the holder case unit 6a, the contact receiver 54 in the ultrasonic transducer 5 can be inserted into the contact unit 20.

When the ultrasonic transducer 5 is inserted into the contact unit 20, a first contact portion 21, a second contact portion 22, a third contact portion 23, and a fourth contact portion 24 of the contact unit 20 and the first contact portion 542, the second contact portion 543, the third contact portion 544, and the fourth contact portion 545 of the contact receiver 54 can be connected, respectively. Responsive to operation of the first switch 8a, the second switch 8b or the third switch 8c, ultrasonic energy or high frequency energy can be output.

While the distal end side Ar1 of the holder 53 is inserted into the proximal end side Ar2 of the contact receiver 54, an O-ring 58 can be arranged between the contact receiver 54 and the holder 53.

In Embodiment 1 described above, the contact receiver 54 can be made as an MID and thus it is possible to reduce a residual stress in molding compared to the conventional insert molding. According to Embodiment 1, reducing the residual stress can reduce the effect caused by distortion, such as cooling distortion, heating distortion, or insertion distortion, which makes it possible to inhibit a mechanical strength in an electric contact portion in an ultrasonic transducer from lowering.

According to Embodiment 1, making the contact receiver 54 into an MID makes it possible to reduce the thickness of the wiring pattern compared to insertion molding and, as a result, it is possible to reduce the size of the contact receiver 54. The size reduction makes it possible to increase freedom in designing.

### Embodiment 2

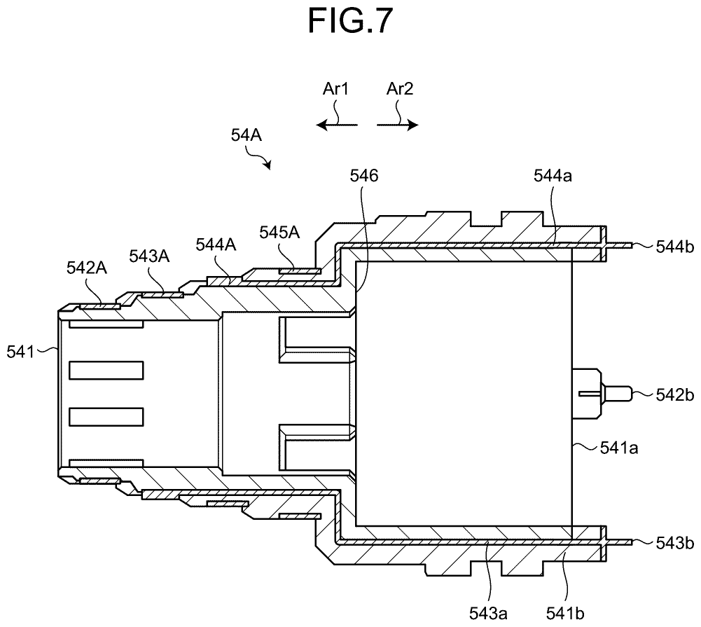

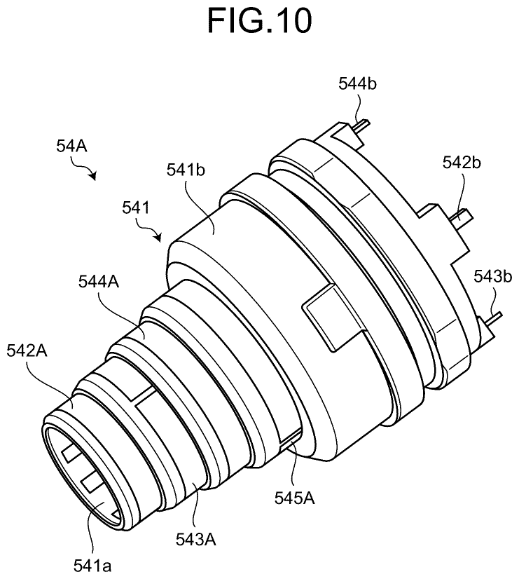

Embodiment 2 of the disclosure will be described next with reference to FIGS. 7 to 10. FIG. 7 illustrates an example of a partial cross-sectional view illustrating a configuration of a contact receiver that an ultrasonic transducer according to Embodiment 2 of the disclosure. In the ultrasonic transducer according to Embodiment 2, each contact portion is also a MID. The same components as those of Embodiment 1 are denoted with the same reference numerals and description thereof will be omitted.

A contact receiver 54A according to Embodiment 2 can include the body 541 made of a resin material that is an electrical insulator and can have a cylindrical (or substantially cylindrical) shape with an outer diameter that increases in steps from the distal end side Ar1 to the proximal end side Ar2.

A first contact portion 542A, a second contact portion 543A, a third contact portion 544A, and a fourth contact portion 545A can be formed in respective step portions of the body 541. The first contact portion 542A, the second contact portion 543A, the third contact portion 544A, and the fourth contact portion 545A can each be formed of an electrically conducting material and can be provided entirely around the respective step portions in the circumferential direction. In the respective contact portions, patterns (first to fourth patterns 542a to 545a) extending to the proximal end side Ar2 can be formed, respectively.

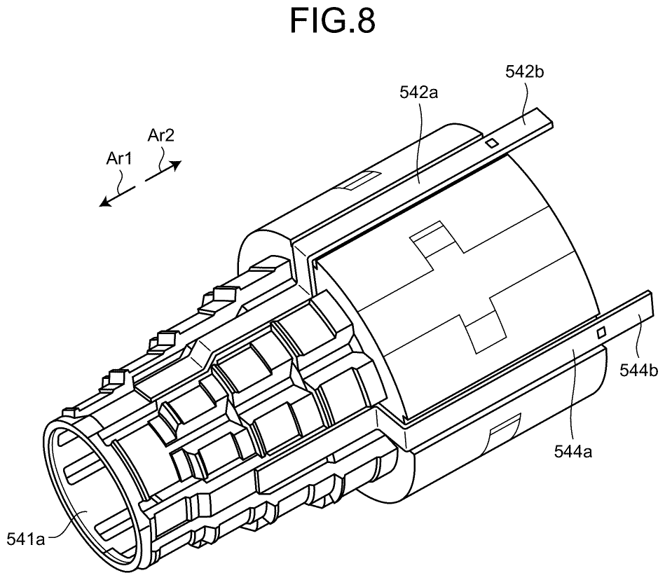

The first contact portion 542A, the second contact portion 543A, the third contact portion 544A, and the fourth contact portion 545A and each pattern can be formed by the above-described pattern formation method (refer to FIGS. 6A to 6C). FIGS. 8 to 10 are diagrams of a method of producing a contact receiver in Embodiment 2 of the disclosure. First of all, a laser can be applied to pattern formation positions on the first portion 541a and patterns (first to fourth patterns 542a to 545a) can be formed by electroless plating in the positions of application of laser (refer to FIG. 8).

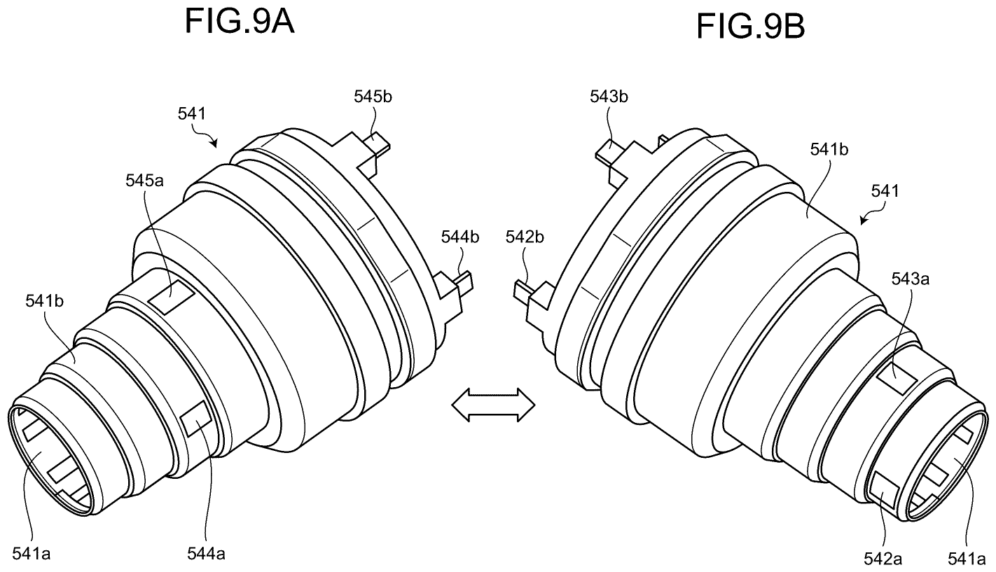

Thereafter, the second portion 541b can be molded on the surface of the first portion 541a (refer to FIGS. 9A and 9B). Note that FIG. 9B is a diagram in which the compact illustrated in FIG. 9A is inverted.

At that time, in the second portion 541b, the patterns can be exposed in positions in which the first contact portion 542A, the second contact portion 543A, the third contact portion 544A, and the fourth contact portion 545A can be formed.

After the second portion 541b is molded, laser can be applied to positions in which the contact portions are formed and the first contact portion 542A, the second contact portion 543A, the third contact portion 544A, and the fourth contact portion 545A can be formed by electroless plating in the positions of application of laser.

In Embodiment 2 described above, the contact receiver 54A can be made as an MID and thus it is possible to inhibit a mechanical strength in an electric contact portion in an ultrasonic transducer from lowering as in Embodiment 1.

Furthermore, in Embodiment 2, in addition to the patterns, the contact portions cab also be made as the MID in the contact receiver 54A and thus it is possible to reduce distortion in the contact portions and further inhibit the mechanical strength in the electric contact portion from lowering.

### Embodiment 3

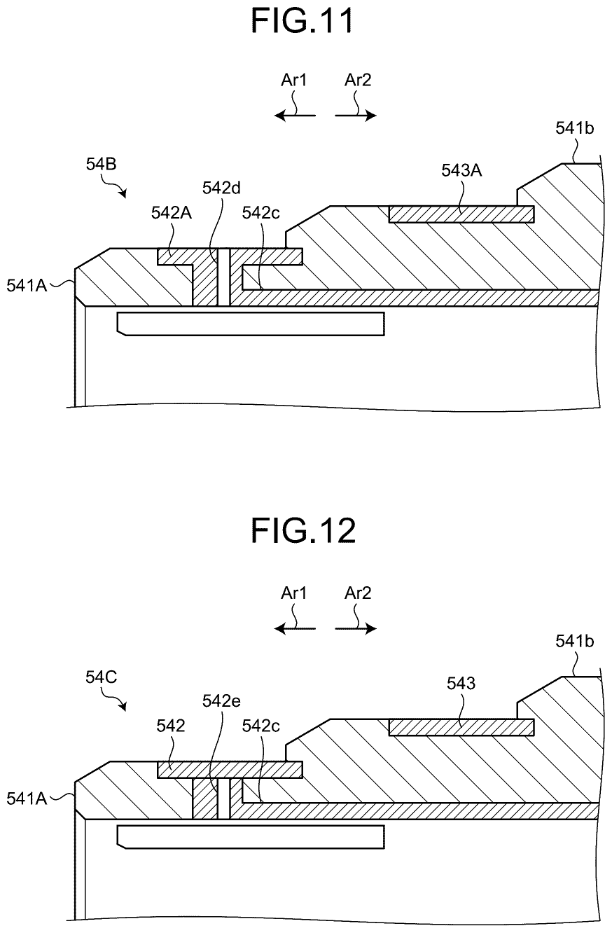

Embodiment 3 of the disclosure will be described next with reference to FIG. 11. FIG. 11 illustrates an example of a partial cross-sectional view illustrating a configuration of part of a contact receiver that an ultrasonic transducer according to Embodiment 3 of the disclosure. The ultrasonic transducer according to Embodiment 3 is different from that of Embodiment 1 in positions of formation of patterns in a contact receiver. The same components as those of Embodiments 1 and 2 are denoted with the same reference numerals and description thereof will be omitted.

A contact receiver 54B according to Embodiment 3 can include a body 541A that is made of a resin material that is an electrical insulator and that can have or be formed in a cylindrical (or substantially cylindrical) shape with outer diameter that can increase in steps from the distal end side Ar1 to the proximal end side Ar2.

The first contact portion 542A, the second contact portion 543A, the third contact portion 544A, and the fourth contact portion 545A can be formed in respective step portions of the body 541A. In the respective contact portions, patterns extending to the proximal end side Ar2 can be formed, respectively. FIG. 11 illustrates only a pattern 542c that is connected to the first contact portion 542A. The pattern 542c will be described as an example below, and each of the patterns connected to the second contact portion 543A, the third contact portion 544A, and the fourth contact portion 545A is the same.

The pattern 542c can extend from the first contact portion 542A to a radial inner circumferential surface side of the body 541A and then can stretch from the proximal end side of the body 541A via an inner circumferential surface of the body 541A.

In the first contact portion 542A and the pattern 542c, a through-hole 542d penetrating in the radial direction can be formed. Note that the position in which the through-hole 542d is formed is not limited to the position illustrated in FIG. 11, specifically, a positon on the proximal end side Ar2 in a longitudinal axial direction, and the position may be a position on the distal end side Ar1 or at the center of an end (end face) of the pattern 542c in the longitudinal axial direction. A configuration in which the through-hole 542d is not formed may be employed.

The first contact portion 542A, the second contact portion 543A, the third contact portion 544A, and the fourth contact portion 545A and each pattern can be formed by the above-described pattern formation method (refer to FIGS. 6A to 6C) on the body 541A. Specifically, a laser can be applied to contact portions and pattern formation positions on the surface of the body 541A (including a hole) and the first contact portion 542A, the second contact portion 543A, the third contact portion 544A, and the fourth contact portion 545A and the patterns can be formed by electroless plating in the positions of application of laser. Accordingly, the contact portions and the patterns can be formed in an integrated manner.

In Embodiment 3 described above, the contact receiver 54B can be made as an MID and thus it is possible to inhibit a mechanical strength in an electric contact portion in an ultrasonic transducer from lowering as in Embodiment 1.

Furthermore, in Embodiment 3, in addition to the patterns, the contact portions can also be made as the MID in the contact receiver 54B and thus it is possible to reduce distortion in the contact portions and further inhibit the mechanical strength in the electric contact portion from lowering.

Furthermore, in Embodiment 3, the body 541A can be molded as one portion and the patterns are made along the inner circumferential surface of the body 541A and accordingly, compared to Embodiment 1 in which the two portions form the BODY, it is possible to further inhibit the mechanical strength in the electric contact portion from lowering.

### Embodiment 4

Embodiment 4 of the disclosure will be described next with reference to FIG. 12. FIG. 12 illustrates an example of a partial cross-sectional view illustrating a configuration of part of a contact receiver that an ultrasonic transducer according to Embodiment 4 of the disclosure. The ultrasonic transducer according to Embodiment 4 is different from that of Embodiment 1 in positions of formation of patterns in a contact receiver. The same components as those of Embodiments 1 to 3 are denoted with the same reference numerals below and description thereof will be omitted.

A contact receiver 54C according to Embodiment 4 can include the body 541A that can be made of a resin material that can be an electrical insulator and that can have or be formed in a cylindrical (or substantially cylindrical) shape and whose outer diameter can increase in steps from the distal end side Ar1 to the proximal end side Ar2.

The first contact portion 542, the second contact portion 543, the third contact portion 544, and the fourth contact portion 545 can be formed in respective step portions of the body 541A. In the respective contact portions, patterns extending to the proximal end side Ar2 can be formed, respectively. FIG. 12 illustrates only the pattern 542c that is connected to the first contact portion 542. The pattern 542c will be described as an example below, and each of the patterns connected to the second contact portion 543, the third contact portion 544, and the fourth contact portion 545 is the same.

The pattern 542c can extend from the first contact portion 542 to a radial inner circumferential surface side of the body 541A and then can stretch from the proximal end side of the body 541A via an inner circumferential surface of the body 541A.

In the pattern 542c, a through-hole 542e penetrating in the radial direction can be formed. Note that the position in which the through-hole 542e is formed is not limited to the position illustrated in FIG. 12. A configuration in which the through-hole 542e is not formed may be employed.

Each pattern can be formed by the above-described pattern formation method (refer to FIGS. 6A to 6C) on the body 541A. Specifically, a laser can be applied to pattern formation positions on the surface of the body 541A (including a hole) and the respective patterns can be formed by electroless plating in the positions of application of laser.

Thereafter, the first contact portion 542, the second contact portion 543, the third contact portion 544, and the fourth contact portion 545 can be fitted to the surface of the body 541 and accordingly the first contact portion 542, the second contact portion 543, the third contact portion 544, and the fourth contact portion 545 can be fitted to the body 541.

In Embodiment 4 described above, the contact receiver 54C can be made as an MID and thus it is possible to inhibit a mechanical strength in an electric contact portion in an ultrasonic transducer from lowering as in Embodiment 1.

In Embodiment 4, the body 541A can be molded as one portion and the patterns are made along the inner circumferential surface of the body 541A and accordingly, compared to Embodiment 1 in which the two portions form the BODY, it is possible to further inhibit the mechanical strength in the electric contact portion from lowering.

The modes for carrying out the disclosure have been described and the disclosure should not be limited by only the above-described embodiments. The pattern formation method (MID) in Embodiments 1 to 4 are an example and it is possible to employ another known method if it is possible to form patterns three-dimensionally on a resin compact.

As described above, the ultrasonic transducer, the ultrasonic treatment tool, and the method of producing an ultrasonic transducer according to the disclosure are useful to inhibit a mechanical strength in an electric contact portion from lowering.

According to the disclosure, it is possible to inhibit a mechanical strength in an electric contact portion from lowering in an ultrasonic transducer.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. An ultrasonic transducer comprising:
a plurality of piezoelectric elements arrayed along a longitudinal direction;
a housing configured to store the plurality of piezoelectric elements; and
a contact receiver fitted to the housing, the contact receiver configured to transmit an electric signal to the plurality of piezoelectric elements, wherein the contact receiver includes:
a body, wherein the body is electrically insulating; and
a wiring pattern electrically connected to a lead provided on the housing, wherein the wiring pattern is formed by three-dimensional plating.

2. The ultrasonic transducer according to claim 1, wherein the body includes:
a first portion having a tubular shape; and
a second portion configured to cover at least a part of an outer surface of the first portion, wherein the wiring pattern is provided between the first portion and the second portion.

3. The ultrasonic transducer according to claim 2, wherein an electrically conducting contact portion has a ring shape, and wherein the electrically conducting contact portion is connected to the wiring pattern on an outer surface of the body.

4. The ultrasonic transducer according to claim 2, wherein an electrically conducting contact portion is formed integrally with the wiring pattern, and wherein the electrically conducting contact portion is provided on the outer surface of the body.

5. The ultrasonic transducer according to claim 1, wherein the body has a tubular shape, and wherein the wiring pattern is provided on an inner circumferential surface of the body.

6. The ultrasonic transducer according to claim 5, wherein an electrically conducting contact portion has a ring shape, wherein the electrically conducting contact portion is connected to the wiring pattern, and wherein the electrically conducting contact portion is provided on an outer surface of the body.

7. The ultrasonic transducer according to claim 5, wherein an electrically conducting contact portion is formed integrally with the wiring pattern, and wherein the electrically conducting contact portion is provided on an outer surface of the body.

8. The ultrasonic transducer according to claim 5, further comprising:
a through-hole penetrating in a direction orthogonal to the longitudinal direction, wherein the through-hole is formed in a first end of the wiring pattern, and wherein the first end is on a first side opposite to a second side on which the wiring pattern is connected to the lead.

9. The ultrasonic transducer according to claim 1, wherein the housing is provided with an ultrasound transducer configured to transmit ultrasonic vibrations from a proximal end side in the longitudinal direction to a distal end side.

10. The ultrasonic transducer according to claim 1, wherein the body is formed using super engineering plastic.

11. An ultrasonic treatment tool comprising:
an ultrasonic transducer; and
a hand piece, wherein the ultrasonic transducer includes:
a plurality of piezoelectric elements arrayed along a longitudinal direction;
a housing configured to store the plurality of piezoelectric elements; and
a contact receiver fitted to the housing, wherein the contact receiver is configured to transmit an electric signal from the hand piece to the plurality of piezoelectric elements, and wherein the contact receiver includes:
a body that is electrically insulating; and
a wiring pattern electrically connected to a lead provided on the housing, wherein the wiring pattern is formed by three-dimensional plating.

12. The ultrasonic treatment tool of claim 11, wherein the body includes:
a first portion having a tubular shape; and
a second portion configured to cover at least a part of an outer surface of the first portion, wherein the wiring pattern is provided between the first portion and the second portion.

13. The ultrasonic treatment tool of claim 11, wherein an electrically conducting contact portion has a ring shape, and wherein the electrically conducting contact portion is connected to the wiring pattern on an outer surface of the body.

14. The ultrasonic treatment tool of claim 13, wherein an electrically conducting contact portion is formed integrally with the wiring pattern, and wherein the electrically conducting contact portion is provided on the outer surface of the body.

15. The ultrasonic treatment tool of claim 11, wherein the body has a tubular shape, and wherein the wiring pattern is provided on an inner circumferential surface of the body.

16. The ultrasonic treatment tool of claim 11, wherein an electrically conducting contact portion has a ring shape, wherein the electrically conducting contact portion is connected to the wiring pattern, and wherein the electrically conducting contact portion is provided on an outer surface of the body.

17. The ultrasonic treatment tool of claim 11, wherein an electrically conducting contact portion is formed integrally with the wiring pattern, and wherein the electrically conducting contact portion is provided on an outer surface of the body.

18. A method of producing an ultrasonic transducer including a plurality of piezoelectric elements that are arrayed along a longitudinal direction, a housing configured to store the plurality of piezoelectric elements, and a contact receiver that is fitted to the housing, the contact receiver being configured to transmit an electric signal to the plurality of piezoelectric elements, the method comprising:
molding a body that is electrically insulating;
forming a wiring pattern by three-dimensional plating on the body, and
electrically connecting the wiring pattern and a lead that is provided on the housing.

19. The method of claim 18, wherein the body has a tubular shape, and wherein the wiring pattern is provided on an inner circumferential surface of the body.

20. The method of claim 19, further comprising:
forming a through-hole penetrating in a direction orthogonal to the longitudinal axial direction, wherein the through-hole is formed in a first end of the wiring pattern, and wherein the first end is on a first side opposite to a second side on which the wiring pattern is connected to the lead.
